(19) United States
(12) Patent Application Publication
(10) Pub. No.: US 2022/0111200 A1
(43) Pub. Date: Apr. 14, 2022

(54) LEAD TEMPLATE AND METHOD OF USE AT A SPINAL TREATMENT SITE

(71) Applicant: SynerFuse, Inc., Minneapolis, MN (US)

(72) Inventors: Michael Park, Excelsior, MN (US); Daniel Craig Oster, Centerville, MN (US); Gregory F. Molnar, Blaine, MN (US); Matthew Hunt, Minneapolis, MN (US); Christopher G. Frank, New Brighton, MN (US)

(21) Appl. No.: 17/462,172

(22) Filed: Aug. 31, 2021

Related U.S. Application Data
(60) Provisional application No. 63/090,493, filed on Oct. 12, 2020.

(51) Int. Cl. A61N 1/05; A61N 1/36

(52) U.S. Cl. CPC A61N 1/0558; A61N 1/36071; A61N 1/36062

(57) ABSTRACT

The present invention provides a lead template for optimal lead placement and spacing during a surgical method and procedure for implantation of a neuromodulation system at a spinal treatment site.

### CROSS-REFERENCE TO RELATED APPLICATIONS

This application claims the benefit of U.S. provisional application No. 63/090,493 filed Oct. 12, 2020 entitled LEAD TEMPLATE AND METHOD OF USE AT A SPINAL TREATMENT SITE, the entire contents of which are incorporated herein by reference.

### BACKGROUND OF THE INVENTION

### Field of the Invention

The present invention relates, either alone or in combination, to the field of spinal fixation for the treatment of spinal instability, and the field neuromodulation for the modulation of chronic pain.

### Description of the Related Art

Neuromodulation for the treatment of chronic spinal pain is a procedure that has been in use for decades. The procedure is generally prescribed to a patient only after they have gone through a spinal procedure that may involve vertebral fusion in an effort to mitigate and/or correct the supposed source of the pain. However, often such spinal procedures do not resolve the pain issues. After weeks, months and perhaps years of continued chronic pain and pain therapy through medications, including opioids, the patient may finally be prescribed neuromodulation for the treatment of chronic pain after failed back surgery.

The prior art neuromodulation systems include an implantable pulse generator (IPG) and one or more neurostimulation leads having a distal portion having one or more electrodes and a proximal portion for electrically coupling the electrodes to the implantable pulse generator. The lead is implanted via a tunneling method, without direct physical or visual access to the target nerve, such that the electrodes are advanced to a position at or near a target nerve and the implantable pulse generator is implanted in a pocket spaced from the target area.

Problems with existing neuromodulation systems include difficulty in achieving satisfactory pain relief due to difficulties with lead placement relative to the nerve target. whether the nerve target is the spinal cord in the case of spinal cord stimulation system or the dorsal root ganglia in the case of dorsal root ganglia stimulation system. Additionally, there is a problem in the prior art of maintaining satisfactory pain relief over time due to lead migration, reduced patient response to a previously efficacious therapy. or due to implantation of the neuromodulation system weeks, months or years after a spinal fixation procedure, or implantation of the neuromodulation system at a different spinal level than the spinal fixation procedure, and other shortcomings that are addressed by the present invention. These problems of the prior art exist both in the case of spinal cord stimulation and dorsal root ganglia stimulation.

Applicant has described a method for the treatment of pain by combining a spinal fixation procedure and a neuromodulation system. U.S. patent application Ser. No. 16/409,616 filed on May 10, 2019 entitled SYSTEM, DEVICES, AND METHODS COMBINING SPINAL STABILIZATION AND NEUROMODULATION. which is herein incorporated by reference in its entirety.

The present application seeks to further improve upon the above referenced matter.

It is an objective of the present invention to provide a method and apparatus for ensuring that there is sufficient anatomical space for the placement of a neurostimulation lead at the target dorsal root ganglia.

It is a further objective of the present invention to provide a method and apparatus for ensuring that there is sufficient anatomical space for the placement of a neurostimulation lead at the same spinal level as a spinal fixation procedure which may include implantation of a spinal fixation system.

It is a further objective of the present invention to prevent the damage and/or migration of the neurostimulation lead during the spinal fixation procedure.

It is yet another objective of the present invention to ensure proper placement of the neurostimulation lead at the target dorsal root ganglia. including facilitating, allowing for, or maintaining direct physical and/or visual access to the target dorsal root ganglia during, at least. the lead placement step of the spinal procedure.

The Figures and the detailed description which follow more particularly exemplify these and other embodiments of the invention.

### BRIEF DESCRIPTION OF THE DRAWINGS

FIG. 1 illustrates a flow diagram of one embodiment of a method for performing a spinal procedure incorporating a lead a template in accordance with the present invention.

FIG. 2 illustrates a cutaway view of one embodiment of a lead template placed over a nerve target in a human anatomy.

FIGS. 3A-3C illustrates cutaway views of embodiments of a lead template placed over a nerve target at a spinal fixational site.

FIG. 4 illustrates a cutaway view of one embodiment of a lead template having radiopaque markers for use in placement over a nerve target.

FIGS. 5A-5D illustrate cutaway views of embodiments of distal ends of a lead template in accordance with the present invention.

FIGS. 6A-6G illustrate cross-sectional views of embodiments of a lead cross section for use with corresponding embodiment of a lead template in accordance with the present invention.

FIGS. 7A-E illustrate cross-sectional views of embodiments of a lead template body in accordance with the present invention.

FIG. 8A illustrates a cross section of an embodiment of a lead template of the present invention.

FIG. 8B illustrates a cross-sectional view of a lead of the present invention.

FIG. 9 is a block diagram of an embodiment of a method for performing a spinal procedure incorporating a lead a template and stylet in accordance with the present invention.

FIG. 10 illustrates a top view of an embodiment of a paddle lead template according to the present invention.

### DETAILED DESCRIPTION OF THE INVENTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

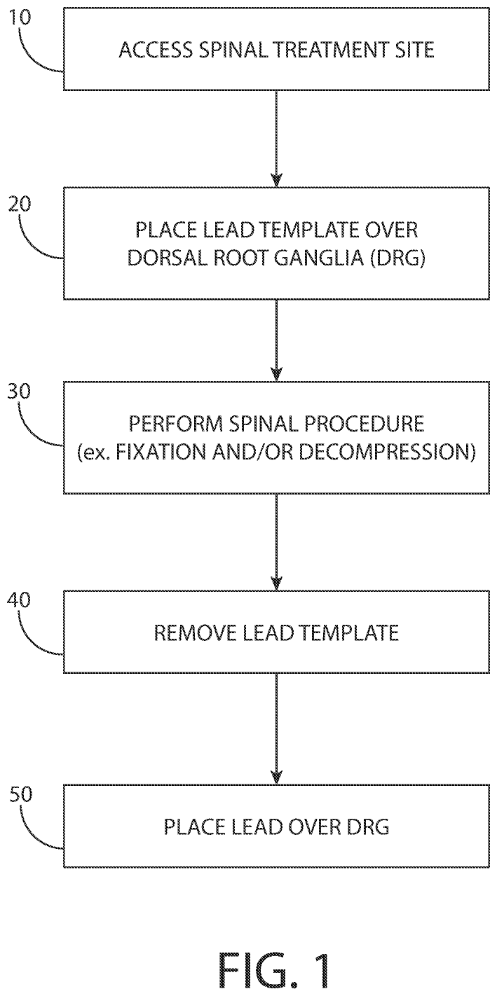

FIG. 1 illustrates a flow diagram of a method for performing a spinal procedure incorporating a lead a template in accordance with the present invention.

The method of performing a spinal procedure in accordance with the present invention includes the step 10 of accessing a spinal treatment site. The spinal treatment site may be access to an open back procedure with full visual and physical access to the dorsal root ganglion (DRG) or, alternatively, minimally invasive procedure.

Once the spinal treatment site has been accessed, a lead a template in accordance with the various embodiment of the present invention is placed over a nerve target in step 20. In the present example, the nerve target is the DRG. Such placing may be done using full visual and physical access created by the access to the spinal treatment site. Further, the nerve target may include any target for treatment of pain or other neurological disease, such nerve targets potentially including but not limited to the spinal cord, specific spinal cord levels, peripheral nerves and the like.

In an open back procedure, the lead template may be positioned over the nerve target with direct visual and physical access to the nerve target. In alternative embodiments, at least a portion of the nerve target is visible by direct visual access by a surgeon performing the procedure, for placement of the lead template. In yet another embodiment, at least a portion of the nerve target may be accessible via direct physical access by a surgeon performing the procedure. during placement of the lead template. In yet another embodiment. a surgeon may not have direct physical or visual access to the target nerve and may place the lead template over the target nerve via CAT. XRAY, fluoroscopy or other indirect navigational techniques.

A spinal procedure may then be performed at the spinal treatment site in step 30, while the template remains positioned over the nerve target. The spinal procedure may be a fixation procedure and/or a decompression procedure and/or any other spinal procedure that may benefit from a lead being placed at the spinal procedure location in accordance with the various embodiments of the present invention.

The lead template may remain positioned over the nerve target for all or a portion of the spinal procedure in step 30. The lead template ensures that sufficient space either exists or has been created by the spinal procedure to allow an active lead to be implanted over the target nerve.

Upon completion of the spinal procedure in step 30. or upon completion of a portion of the spinal procedure that ensures there is. or will be, sufficient space over the nerve target for placement of an active lead, the lead template is removed from the spinal treatment site in step 40.

A lead may then be placed over the spinal treatment site in step 50. The lead may be any lead sufficient to deliver an electrical stimulation therapy to the nerve target. Such leads generally having one or more electrodes at a distal portion thereof, electrically coupled to a conductor that extends to a proximal portion of the lead. The proximal portion of the lead is then electrically coupled to an implantable pulse generator which provides an electrical stimulation signal to the electrodes. Various lead designs are common in the art and may be used in accordance with the present invention, including but not limited to percutaneous leads, paddle leads, segmented leads and other lead designed. depending upon the particular therapeutic purpose.

Various embodiments of a lead template are provided below, in accordance with the various embodiments of the present invention.

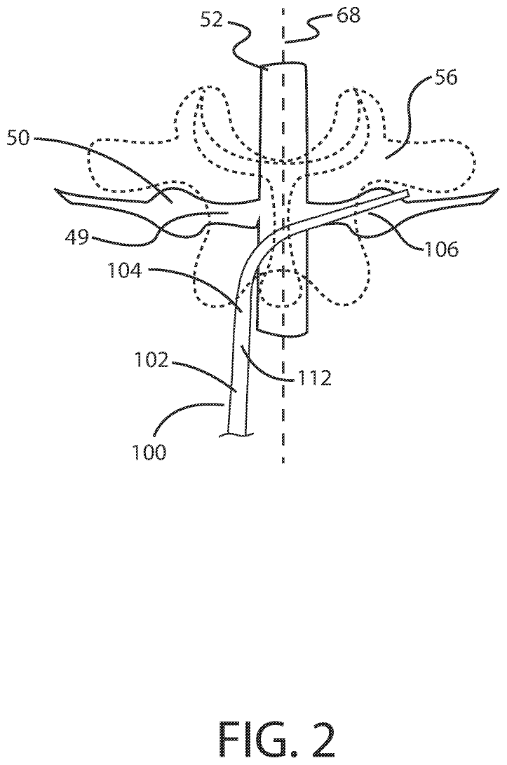

FIG. 2 is an illustration of a lead template 100 placed over a nerve target in a human anatomy. A spinal cord 52 is shown along with a dorsal root 49 extending from the spinal cord 52 to a dorsal root ganglion 50. A vertebrae 56 is shown at a first vertebral level 56 above the dorsal root ganglia (DRG). An exemplary lead template 100 is shown, having a lead template body 112 defined by a proximal portion 102. central portion 104 and a distal portion 106. The proximal portion 102 of the lead template 100 is generally parallel to the midline 68 of the spinal cord 52 and the distal portion 106 extends generally lateral to the midline 68 of the spinal cord 52. over the nerve target (dorsal root ganglion). As shown, the lead template 100 is arranged in a medial-to-lateral placement of the lead template 100 over the nerve target 50. The central portion 104 of the lead template 100 is curved to enable such placement. The lead template body 112 may be made of a malleable material that can be shaped according to the needs of a particular spinal treatment procedure. or alternatively may have a preformed shape as determined suitable for a particular type of spinal treatment procedure and/or nerve target combination. Any suitable material or combination of materials may be used in consideration of malleability. rigidity. biocompatibility such as by way of non-limiting example, medical grade silicon having the dimensions and stiffness/malleability properties for a particular procedure in accordance with the present invention.

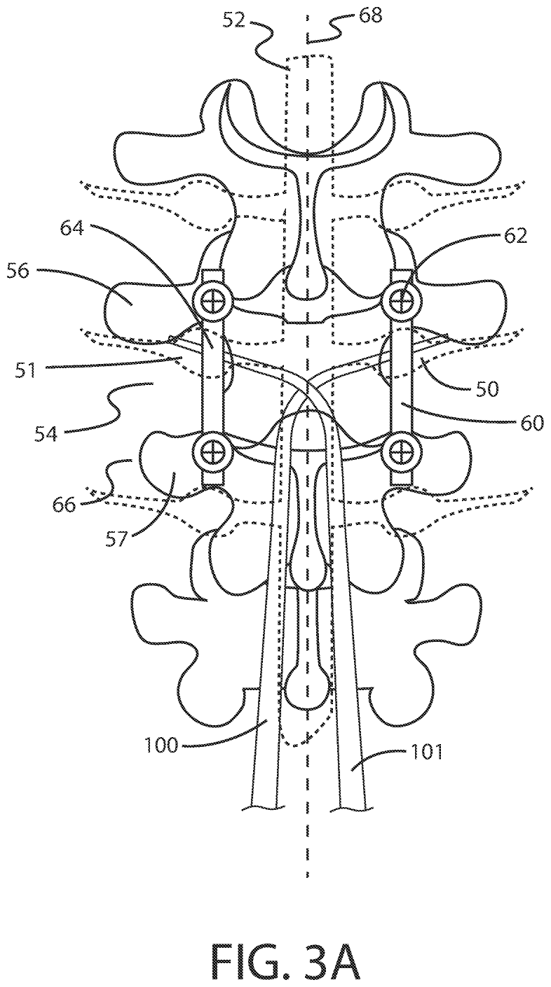

FIGS. 3A-3C illustrate a first and second lead template 100, 101 placed over a first and second nerve target 50, 51 at a spinal treatment site 54. A spinal fixational device 64 is implanted at the spinal treatment site 54. The spinal treatment site 54 is defined by a space between a first vertebral level 56 and a second vertebral level 66. As shown, a portion of the spine has been removed at the level of the spinal treatment site. The target nerve 50, 51 may be defined as one or both of the DRG between the two vertebral levels 56, 66 at the spinal treatment site.

The fixation device 64 may include a pair of fixational rods 60 each extending along opposing sides of the spinal cord 52, substantially in parallel with the midline 68 of the spinal cord 52. Each of the fixation rods 60 having first and second ends. each end fixated to a corresponding first and a second vertebrae 56, 57. via corresponding first and the second fixation screws 62.

In FIG. 3A, a first exemplary lead template 100 is shown having a distal portion positioned over a first dorsal root ganglion 50. A second exemplary lead template 101 is shown having a distal portion positioned over a second dorsal root ganglion 51. The first and second lead template 100, 101 cross over the spinal cord midline 68 before approaching the nerve target 50, 51 on opposing sides of the spinal cord.

In FIG. 3B, the second exemplary lead template 102 is shown positioned over the second nerve target 51 without crossing the midline 68 of the spinal cord. In FIG. 3C. the first and second lead templates 101, 102 are shown having a distal portion that forms a large curve as it approaches the nerve target 50, 41. It is understood that the lead template(s) 101 and/or 102 may be positioned to approach the target nerve using any of a variety of curvatures of approach and or shaped approaches defined by the distal portion, central portion and proximal portion of the lead template. It is understood that the present embodiment is merely illustrative and that in various embodiments, one or more lead templates may be used, one or more lead template may cross the spinal cord midline or, either with or without crossing over each other, or undertake any number of arrangements as determined necessary or beneficial for performing the spinal fixation procedure and/or lead placement step of the spinal fixation procedure. These alternative arrangements may include but are not limited to one or more lead templates. either alone or in combination with other lead template placements, being positioned over a nerve target at a vertebral level above or below the vertebral levels defining the spinal treatment site.

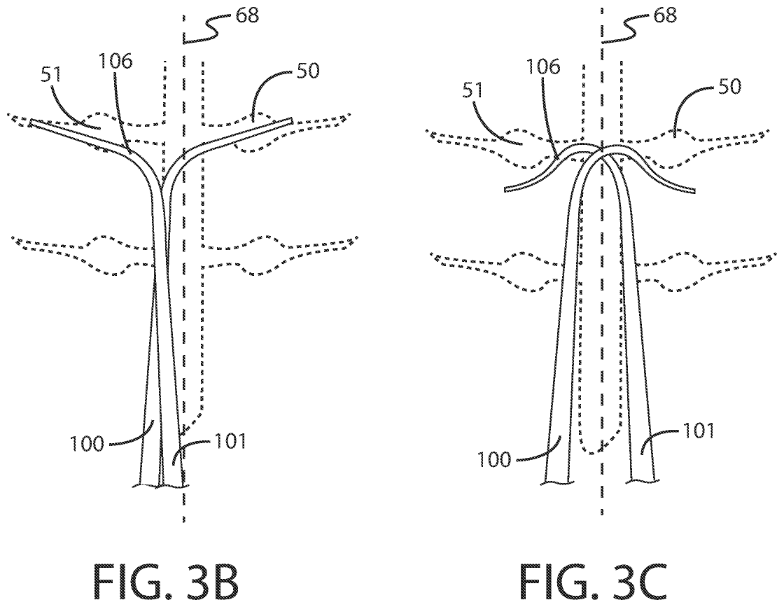

FIG. 4 is an illustration of an exemplary lead template 100 further comprising radiopaque markers 200, 202, 204 for use in placement over a nerve target, e.g., a DRG, 50. The lead template 100. as shown, has a distal portion 106 with one or more radiopaque markers 200, 202, 204 on a surface of the lead template body. A pair of boundary radiopaque markers 200, 204 provide a visual guide for placing the lead template over a nerve target. A central radiopaque marker 202 may be positioned between the two boundary markers 200, 204 to enable precise positioning and/or placement of the lead template 100 over the nerve target 50. Placement of the lead template 100 may be performed under direct visual and or physical access, or through the use of indirect visualization such as fluoroscopy. X-Ray, CTscan visualization. In the case of direct visual access, the radiopaque markers 200, 202, 204 may also visually and/or physically distinct by color, size, shape. shade, grooves, ridges and the like.

As shown, a dorsal root ganglion 50 may be a nerve target and the boundary markers 200, 204 me extend approximately the length of the dorsal root ganglion 50 and the central marker 202 may be positioned over a central portion of the dorsal root ganglia or otherwise positioned over a preferred location of the dorsal root ganglion or other nerve target.

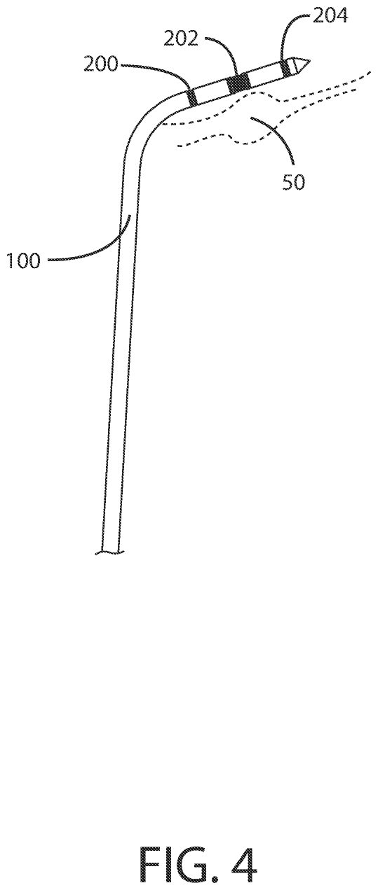

FIGS. 5A-5D or illustrations of the various embodiments of distal ends of a lead template in accordance with the various embodiments of the present invention. FIG. 5A shows a distal end 300 of a lead template with a pointed distal tip having a beaded end 310. FIG. 5B shows a distal end 302 of a lead template with a pointed distal tip having a rounded end 312. FIG. 5C shows a distal end 304 of a lead template with a hollow lumen 314 extending through at least a portion of the lead template body. FIG. 5D shows a distal end 306 of a lead a template with a hollow lumen 316 extending through at least a portion of the lead to template body defining a cannula tip 318.

These embodiments shown in FIGS. 5A-5D, and additional combinations and modifications, are anticipated by the present invention for providing a distal end of a lead template that allows for the advancement of the lead template through tissue in order to access a nerve target. Even in the case where an implanting surgeon has direct physical and visual access to a nerve target. the tissue spacing above the nerve target may not be sufficient to allow placement of the lead template without the use of a distal tip designed to clear the tissue space above the nerve target. Additionally, the embodiments that incorporate a hollow lumen in the lead body allow for a stylet to be used in the placement of the lead template, including the advancement of the lead template to the nerve target and/or defining a shape of the lead body via a malleable stylet extending through the lumen.

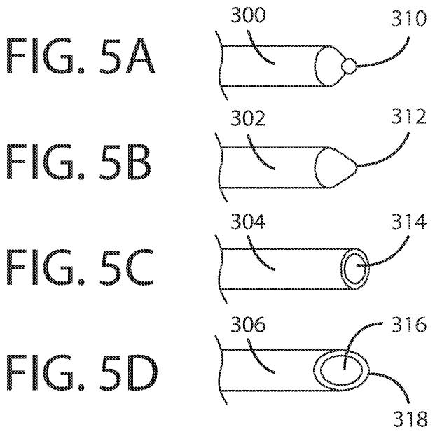

FIGS. 6A-6G illustrate various embodiments of a lead in cross-section for use with a corresponding embodiment of a lead template in accordance with various embodiments of the present invention.

FIG. 6A shows a cutaway of a distal portion of an exemplary lead body. revealing a cross-section of the lead 400, as shown in more detail in FIG. 6B. The lead body includes a number of conductor lumens 404 with conductors extending therethrough along the length of the lead body.

The lead body further includes a lead template lumen 402 for slideably receiving the lead template therein.

FIGS. 6C-6E are alternative embodiment of a lead body having a lead a template lumen 402. In accordance with various embodiments, the lead template can be placed over the nerve target and then the lead 400 is positioned over the nerve target by advancing the lead along the length of the lead template 100, where the lead template is received within the lead template lumen 402 of the lead 400 itself. Various embodiments allow for the lead template 100 to remain within the lead template lumen 402 during implant of the lead 400 or, alternatively, the lead template 100 can be removed from the lead template lumen 402 after the lead has been placed or positioned over the nerve target.

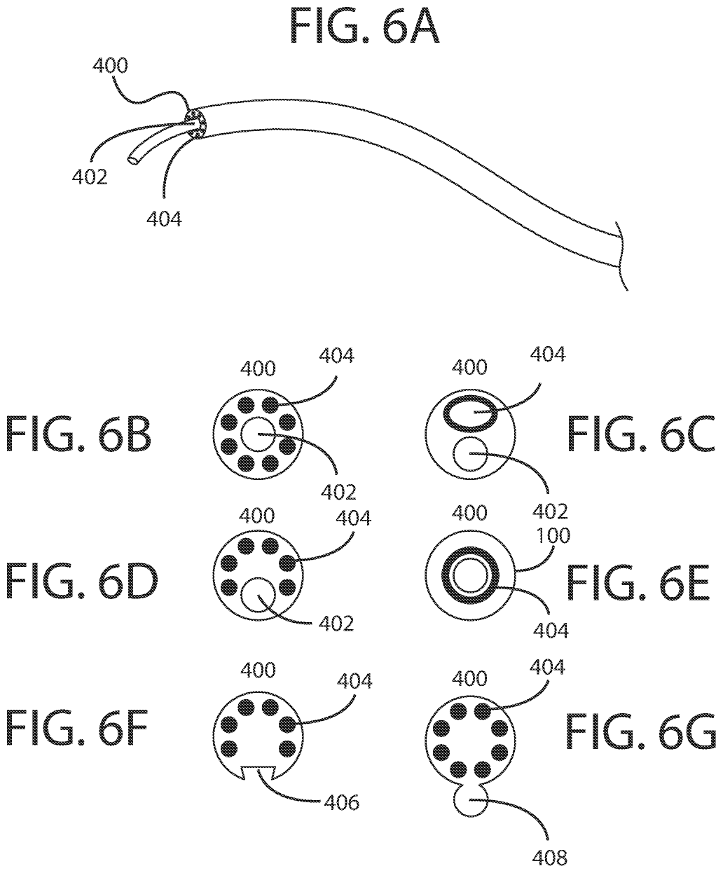

FIG. 6F shows a lead cross-section having a notched receiving portion 406 configured to slideably receive the extension portion 500 of the lead template body 112 shown in FIG. 7A. FIG. 6G shows a lead cross-section having a rail extension 408 configured to be slideably received in the corresponding rail receptacle 502 of the lead template body 112 cross-section shown in FIG. 7B. In the lead-lead template combinations shown in FIGS. 6F-6G and FIGS. 7A-7B. the lead and template are slidably coupled to each other. When a lead template is placed over a nerve target, the slidable coupling allows for the slideable placement of the lead over the nerve target and the corresponding slidable removal of the lead template from the spinal treatment site, while still leaving the lead in position over the nerve target.

FIGS. 7C-7E illustrate a cross section 600 of a lead a template body in accordance with various embodiments of the present invention wherein the lead template body is curved to define a plenum 602 extending along an axial length of the lead body 112. The lead template 100 may be pre-shaped and/or pre-bent along an axial length such that a distal portion of the lead template is curved or angled with respect to the proximal portion of the lead template. Additionally, the lead template may be configured such that the plenum 602 faces away from the exemplary nerve target 50. as shown in FIG. 7D, or the plenum 602 faces toward the nerve target, as shown in FIG. 7E. In either case, a lead can be advanced and/or received within the plenum 602 for placement of the distal portion of the lead over the nerve target. The lead template may then be removed from the spinal treatment site or, alternatively, may remain at the spinal treatment site for preventing or minimizing lead migration.

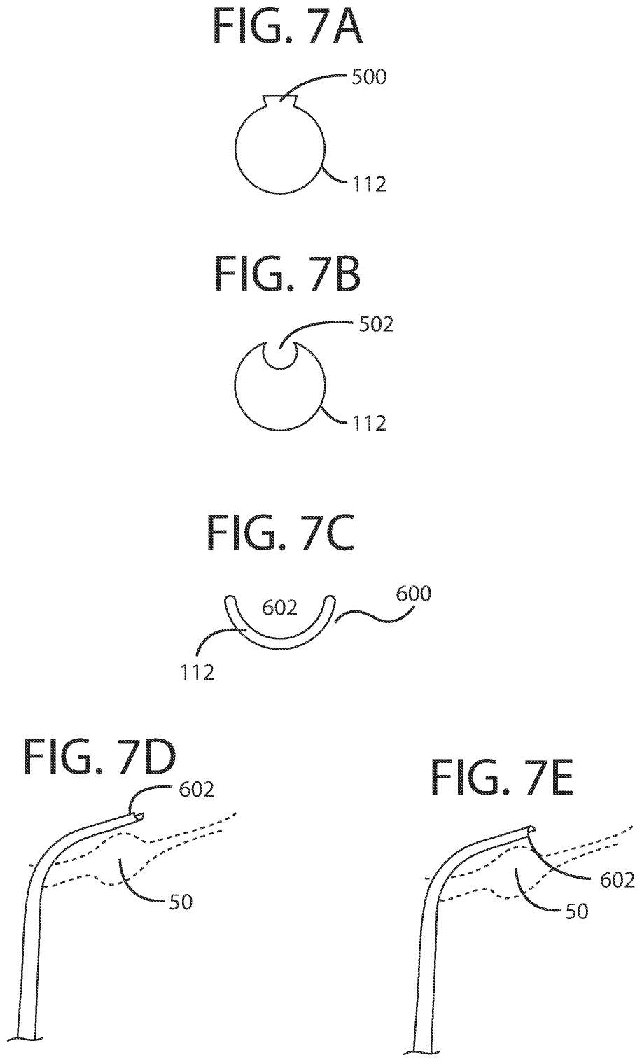

FIG. 8A illustrates a cross section of a lead template with a lead template body 112 defining a lumen 700 for slideably and removably receiving a stylet. FIG. 8B illustrates a cross section of a lead with a lead body 702 defining a lumen 704 for slideably and removably receiving a stylet.

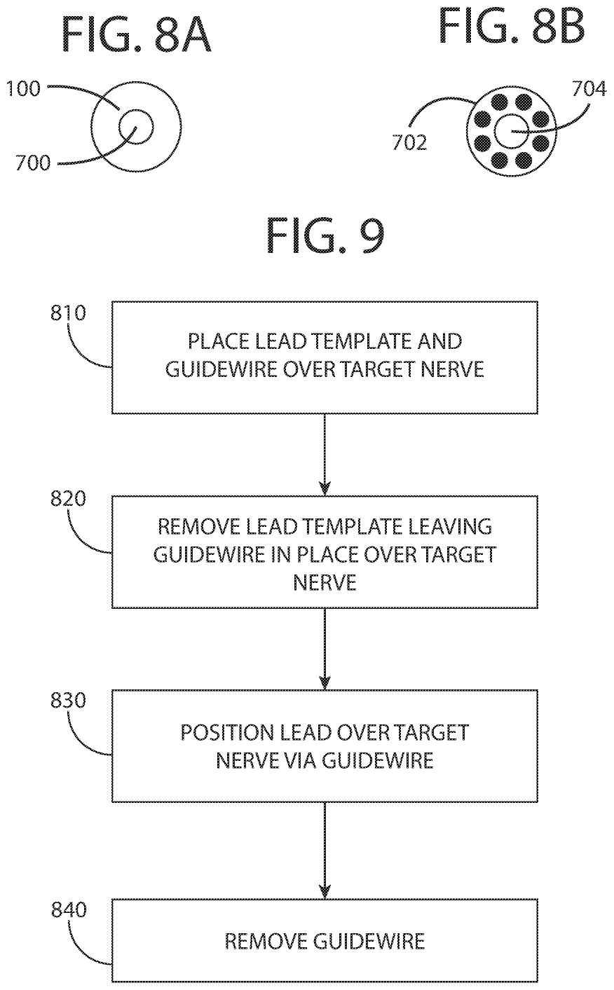

FIG. 9 is a block diagram of an exemplary method for performing a spinal procedure incorporating a lead template, a lead and stylet as described with reference to FIGS. 8A and 8B. In step 810, the lead template of FIG. 8A can be placed directly on a nerve target—specifically, so that the distal portion of the lead template is positioned over the nerve target, for example under direct physical and visual access. For such placement, the stylet may be pre-placed within the lumen and remain in the lumen during all or a portion of the spinal procedure. In step 820. the lead template is slideably removed from the treatment site, while leaving a distal portion of the stylet positioned over the spinal treatment site. In step 830. the lead from FIG. 8B is then slideably positioned over the nerve target by advancing the lead body along the stylet received within the lead lumen such that a distal portion of the lead, having one or more electrodes for electrically stimulating the nerve target, are positioned over the nerve target. In step 840, the stylet is slideably removed from the lead lumen and spinal treatment site. leaving the distal portion of the lead in place, positioned over the target nerve. Alternatively, in some embodiments, the stylet may have a predefined curve and/or shape and may remain in the lead in order to maintain the shape and position of the lead with respect to the spinal treatment site, patient anatomy and/or nerve target, potentially reducing or mitigation lead migration.

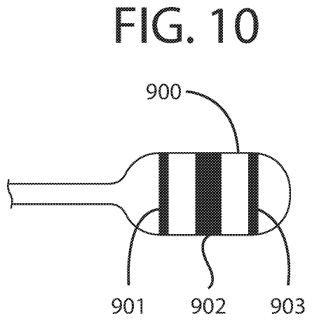

FIG. 10 is an illustration of an embodiment of a paddle lead template 900 with radiopaque markings 901, 902, 903. As shown in FIG. 10, the lead template 900 may have a distal portion shaped (shown as the enlarged section bearing markings 901, 901, 903) to the dimensions and cross-sections of a predetermined paddle lead corresponding to a nerve target of interest and corresponding to the shapes and dimensions of the paddle lead that is intended to be implanted over the nerve target. The paddle lead template 900 further incorporates radiopaque markings 901, 902, 903 corresponding to sizing and spacing that facilitates placement of the lead template over a nerve target under direct or indirect visual and/or physical access to the target nerve.

In the various embodiments described herein, the lead template may have a general size category of small, medium or large as relating to the corresponding size of the lead that is to be implanted at the nerve target. The lead template may differ slightly in size, such as diameter or length, from the lead that is to be implanted. For example, the lead template may be slightly larger in diameter than the implanted lead for the purpose of ensuring enough anatomical space is maintained and/or created for the lead to be placed over the nerve target. Alternatively, the lead template may be slightly smaller in diameter for the purpose of ensuring the later implanted lead will be prevented from migration and/or minimizing potential migration from an ideal placement location with respect to a target nerve.

Additionally. the various embodiments provide the benefit of ensuring that there is space for lead placement while also ensuring that the lead is not damaged during a spinal procedure such as by placement of a spinal fixation device, bone and tissue removal, damage from a scalpel, plasma blade, cautery tool, and the like. Likewise, the lead template prevents energy delivery to the target nerve during the spinal procedure. Once the spinal procedure has been performed, or those steps most likely to potentially damage a lead, the lead template can be removed and/or replaced with the lead.

In addition to the above, the contents of U.S. patent application Ser. No. 16/519,320 filed on Jul. 23, 2019 entitled METHOD FOR IMPLANTING A NEUROMODULATION SYSTEM AT A SPINAL TREATMENT SITE and provisional patent application 62/702,867. filed Jul. 24, 2018 and entitled METHOD FOR IMPLANTING A NEUROMODULATION SYSTEM AT A SPINAL TREATMENT SITE are hereby incorporated by reference. The contents of U.S. patent application Ser. No. 16/665,525 filed on Oct. 28, 2019 entitled SYSTEMS. DEVICES AND METHODS FOR IMPLANTABLE NEUROMODULATION STIMULATION and provisional patent application 62/752,223 filed on Oct. 29, 2018 entitled IMPLANTABLE NEUROMODULATION SYSTEM AND KIT are hereby incorporated by reference. The contents of U.S. patent application Ser. No. 16/409,616 filed on May 10, 2019 entitled SYSTEM, DEVICES. AND METHODS COMBINING SPINAL STABILIZATION AND NEUROMODULATION and provisional patent application 62/670,034, filed May 11, 2018, entitled “Method and Apparatus to Deliver Neuromodulation to the Spine” are hereby incorporated by reference.

While the embodiments of the present invention described herein are illustrative, it is understood that they are merely exemplary and that various additional lead template embodiments may be formed of combinations and/or modifications of various embodiments and variations including but not limited to various shapes. profiles, materials, dimensions, cross-sections and combinations thereof. It is understood that such additional embodiments fall within the scope and spirit of the present invention.

The descriptions of the embodiments and their applications as set forth herein should be construed as illustrative, and are not intended to limit the scope of the disclosure. Features of various embodiments may be combined with other embodiments and/or features thereof within the metes and bounds of the disclosure. Upon study of this disclosure, variations and modifications of the embodiments disclosed herein are possible and practical alternatives to and equivalents of the various elements of the embodiments will be understood by and become apparent to those of ordinary skill in the art. Such variations and modifications of the embodiments disclosed herein may be made without departing from the scope and spirit of the invention. Therefore, all alternatives, variations, modifications, etc., as may become to one of ordinary skill in the art are considered as being within the metes and bounds of the instant disclosure.

1. A method for implanting a lead at a spinal treatment site:
a. identifying a target spinal level for a spinal fixation procedure;
b. accessing the spinal level for implantation of a spinal fixation device;
c. positioning a lead template at a target DRG accessible from the spinal level of the spinal fixation device, the lead template not being capable of delivering a neurostimulation signal;
d. leaving the lead template over at least a portion of the DRG during at least a portion of the spinal fixation procedure; and
e. removing the lead template and replacing the lead template with a neurostimulation lead capable of delivering a neuromodulation signal to a nerve target
f. placing the neurostimulation lead in therapeutic proximity to the target DRG.

2. The method according to claim 1 wherein the lead template is configured to maintain a spacing between the DRG and tissue at the treatment site sufficient to ensure that sufficient space will remain for a neurostimulation lead to be placed over the corresponding DRG upon completion of the spinal fixation procedure.

3. The method according to claim 1 wherein the lead template has a distal portion configured to create space near the target DRG to allow a neurostimulation lead to be placed over the target DRG.

4. The method according to claim 3 wherein the lead template has a distal portion configured to create space tissue space near the target DRG sufficient to allow direct visual and physical access to the target DRG.

5. The method according to claim 4 wherein the lead template is removed and a neurostimulation lead is placed over the target DRG using direct visual and physical access to the target DRG.

6. The method according to claim 5 wherein the target DRG is at the same spinal level as the spinal fixation procedure.

7. The method according to claim 5 wherein the target DRG is one level above or below the spinal level of the spinal fixation procedure.

8. A lead template comprising:
a distal portion configured to allow placement over a target DRG during a spinal fixation procedure;
an engaging element configured to engage with a neurostimulation lead, wherein the lead template is not configured to deliver a neuromodulation signal to the target DRG by itself but is configured to deliver a neuromodulation signal to the target DRG when engaged with the neurostimulation lead.

9. The lead template from claim 8 wherein the engaging element comprises a groove along a distal portion of the template and configured to receive a neurostimulation lead having a corresponding mating portion configured to be slidably inserted into the groove of the lead template.

10. The lead template from claim 8 where the engagement element comprises a stylet wherein the neuromodulation lead is configured to be slidably placed over the stylet in order to provide a neuromodulation signal to a target nerve.

11. The lead template from claim 8 wherein the lead template comprises one or more radiopaque markers configured for visualization under fluoroscopy or x-ray.

12. The lead template from claim 11 wherein the radiopaque markers are spaced to reflect a location of a target dorsal root ganglia when the lead template is positioned at a spinal treatment site.

13. The lead template from claim 12 wherein the engaging element is a lumen extending through the lead template capable of receiving a neurostimulation lead therein.

14. The lead template from claim 13 wherein a portion of the lead template includes at least one window configured to allow a neurostimulation signal to reach a target dorsal root ganglia when a neurostimulation lead is positioned within the lumen.
